(19) United States
(12) Patent Application Publication
(10) Pub. No.: US 2020/0108165 A1
(43) Pub. Date: Apr. 9, 2020

(54) Handheld and rechargeable battery operated device invented to eliminate the bad odor produced by human flatulence and feces by ignition.

(71) Applicants: Carlos Manuel Poventud-Estrada, Fort Bragg, NC (US); Paola Aliene Escoriaza-Santana, Fort Bragg, NC (US)

(72) Inventors: Carlos Manuel Poventud-Estrada, Fort Bragg, NC (US); Paola Aliene Escoriaza-Santana, Fort Bragg, NC (US)

(21) Appl. No.: 16/152,957

(22) Filed: Oct. 5, 2018

(51) Int. Cl. A61L 9/16; H05B 1/02

(52) U.S. Cl. CPC A61L 9/16; H05B 1/0252

(57) ABSTRACT

Handheld and rechargeable battery operated device that eliminates the bad odor coming from human flatulence and feces by ignition.

### CROSS REFERENCE TO RELATED APPLICATION (IF ANY)

NONE

### STATEMENT IF FEDERALLY SPONSORED RESEARCH OR DEVELOPMENT (IF ANY)

NONE

The names of the parties to a joint research agreement if the claimed invention was made as a result of activities within the scope of a joint research agreement: NONE

Reference to a “Sequence Listing,” a table, or a computer program listing appendix submitted on a compact disc and an incorporation by reference of the material on the compact disc. The total number of compact disc including duplicates and the files on each compact disc shall be specified: NONE

### BACKGROUND OF THE INVENTION

In certain instances, human flatulence and feces generate gases with characteristic disagreeable odors (e.g. H₂S, C₂H₆S, etc.). Remedies to eliminate this stench vary from lighting a match or candle to chemical solutions for the toilet or aerosols. Nonetheless, there is no electronic device ever designed or built for the purpose of eliminating these gases by ignition.

### BRIEF SUMMARY OF THE INVENTION

A device that is handheld and rechargeable battery operated invented to eliminate the bad odor coming from human flatulence and feces by ignition.

### BRIEF DESCRIPTION OF THE SEVERAL VIEWS OF THE DRAWING (IF ANY)

There are two figures in the Drawings annex:

FIG. 1 Schematic representation of the device and

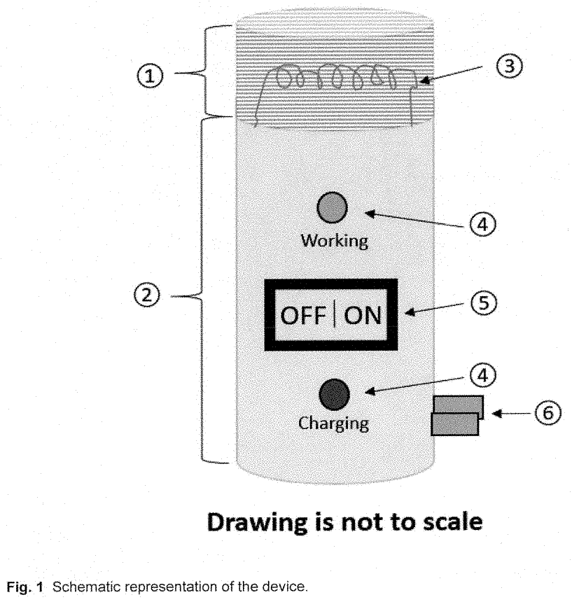

FIG. 2 Internal circuit diagram of the device.

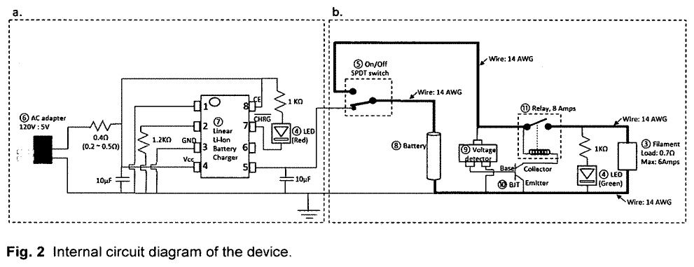

### LEGEND AND SPECIFICATIONS (DESCRIPTION) FOR THE DRAWINGS

a. This portion of the circuit and components are commercially available.

b. This portion is a custom-made circuit claimed hereto as part of the invention in Claim no. 2.

c. All wiring is 22 AWG unless specified.

d. All values for the components shall be within a 10% tolerance.

\textcircled{1} Cap: all plastic ventilation grid.

\textcircled{2} Body: all solid plastic.

\textcircled{3} Filament (located inside the cap): Equal of similar to Nichrome 22 gauge, 80 wire, 0.7 Ω.

\textcircled{4} Light emitting diode (LED).

\textcircled{5} ON/OFF switch: single pole, double throw switch.

\textcircled{6} AC adapter input: 120V, ˜50/60 Hz, 0.2 A, two-prong connector; DC output: 5V, 1.0 A

\textcircled{7} Linear Li-ion battery charger: equal or similar to TC4056A.

\textcircled{8} Battery: 18650 integrated, 3.6V, 3000 mAh, 15 A high discharge capacity. Equal or similar to Samsung INR18650-30Q.

\textcircled{9} Voltage detector: Equal or equal to Microchip TC-54 CMOS Voltage Detector for battery applications.

\textcircled{10} Bipolar Junction Transistor (BJT) NPN: Equal or similar to On Semiconductor catalog number MPSA05RA.

\textcircled{11} Relay switch: Equal or similar to Omron Catalog Number G6RL-1A-DC3, 10 A, 3 VDC.

### DETAILED DESCRIPTION OF THE INVENTION

A device that is handheld, mobile, cordless, rechargeable battery operated, silent and flameless which contains an electrically heated filament calibrated to ignite the gases that generate the bad odor coming from human flatulence and feces. This device is intended to be used anywhere that can be safely operated (e.g. bathroom, car, office, etc.) to eliminate the bad odors. The user turns ON the switch and the filament will reach the operational temperature in an average of 15 s. The gases with the bad odor diffuse through a ventilation grid and are eliminated by the filament via ignition. The user can hold the device's body safely while the filament is energized. The plastic ventilation grid will preclude any reasonable size object to come in direct contact with the filament.

SAFETY WARNING: The surface of the filament reaches temperatures beyond 200° C. that can ignite vapors (e.g. fuel vapor, etc.).

Operational description (also see Table 1):

Basic User Operation:
a. When the device is ON, the WORKING Light Emitting Diode (LED, green) will be on and the filament will begin to ignite the gases upon reaching ca. 250° C. (an average of 15 s). If the WORKING LED remains off or extinguishes, it indicates that the battery needs to be recharged.

Charging Operation:
a. The device can only recharge while is OFF. If connected to an outlet while is OFF, the CHARGING LED (red) will be on indicating is recharging. The CHARGING LED will extinguish once the battery reaches its maximum capacity or when the device is disconnected from the outlet.
b. If the device is connected to an outlet and turned ON, it will operate from the battery (NEVER from the outlet).

### NOTES:

1. The filament will never be energized while charging.
2. Once the battery reaches a level which cannot sustain the required temperature on the filament, the system will automatically disconnect the filament. Then, the WORKING LED will flash intermittently to indicate that the device needs to be turned OFF and recharged.

TABLE 1
The device operational states and indicating LEDs.

| | ON/ | | | | |
|---|---|---|---|---|---|
| | OFF | Connected | Red LED | Green LED | |
| State | Switch | to outlet | (Charging) | (Working) | Filament |
| 1 | OFF | No | off | off | Not energized |
| 2 | OFF | Yes | on | off | Not energized |
| 3* | OFF | Yes | off | off | Not energized |
| 4 | ON | No | off | on | Energized |
| 5 | ON | Yes | off | on | Energized |
| 6** | ON | Yes | off | off | Not energized |
| 7** | ON | Yes | off | Intermittent | Not energized |

*The device is fully charged.
**The device needs charge. The switch needs to be in the OFF position to recharge.

1. The device is the only electronic device ever created to eliminate via ignition the gases that generate the bad odor coming from human flatulence and feces.

2. The device is handheld, mobile, cordless, rechargeable battery operated, silent and flameless. It contains an electrically heated filament calibrated to ignite the gases that generate the bad odor coming from human flatulence and feces.

3. This device is controlled with a custom-made electronic circuit to operate any load that requires up to 10V and up to 8 Amps with a Li-ion rechargeable battery. It automatically shuts off the load when the battery reaches a preset voltage (e.g. 3V) and recharges it while connected to an outlet while is OFF.
